(19) United States
(12) Patent Application Publication
(10) Pub. No.: US 2026/0026872 A1
(43) Pub. Date: Jan. 29, 2026

(54) VERTEBRAL BODY TREATMENT DEVICE WITH SIDE PORT

(71) Applicant: Relievant Medsystems, Inc., Redwood City, CA (US)

(72) Inventors: Benjamin David Parker, Sunnyvale, CA (US); Neil Shigeo Sasaki, Edina, MN (US); Michael Makay, Stafford, VA (US)

(21) Appl. No.: 19/279,521

(22) Filed: Jul. 24, 2025

Related U.S. Application Data
(60) Provisional application No. 63/676,231, filed on Jul. 26, 2024.

(51) Int. Cl. A61B 18/14; A61B 17/00; A61B 18/00; A61B 18/12

(52) U.S. Cl. CPC A61B 18/1477; A61B 2017/00367; A61B 2018/00077; A61B 2018/0044; A61B 2018/00577; A61B 2018/00773; A61B 2018/126; A61B 2018/1467

(57) ABSTRACT

A medical device for treating a basivertebral nerve in a vertebral body of a patient includes an elongate shaft defining a central channel with a port proximate the distal end. The shaft further comprises a sharpened tip for piercing the vertebral body and a handle including an actuator. A channeling member is disposed within the central channel and is movable to a position wherein the distal end of the channeling member is exterior of the port. A tracking device is disposed within the central channel of the shaft and surrounding at least a portion of the channeling member and is movable to a position in which the distal end of the tracking device is disposed exterior of the port. First and second electrodes are carried by one or both of the channeling member and the tracking device and are configured to deliver radiofrequency energy at the treatment location.

### CROSS REFERENCE TO RELATED APPLICATIONS

The present application claims priority to U.S. Provisional Patent Application No. 63/676,231, filed Jul. 26, 2024, the disclosure of which is incorporated herein in its entirety.

### TECHNICAL FIELD

Described herein are various implementations of systems and methods for modulating tissue. More specifically, the disclosure relates to systems for accessing and treating locations within vertebral bodies, for example, by ablating nerves or other tissue within or surrounding a vertebral body to treat chronic lower back pain.

### BACKGROUND

Back pain is a very common health problem worldwide and is a major cause for work-related disability benefits and compensation. At any given time, low back pain impacts nearly 30% of the US population, leading to 62 million annual visits to hospitals, emergency departments, outpatient clinics, and physician offices. Back pain may arise from strained muscles, ligaments, or tendons in the back and/or structural problems with bones or spinal discs, including vertebral endplate degeneration or defects (e.g., pre-Modic changes). The back pain may be acute or chronic. Existing treatments for chronic back pain vary widely and include physical therapy and exercise, chiropractic treatments, injections, rest, pharmacological therapy such as opioids, pain relievers or anti-inflammatory medications, and surgical intervention such as vertebral fusion, discectomy (e.g., total disc replacement), or disc repair. Existing treatments can be costly, addictive, temporary, ineffective, and/or can increase the pain or require long recovery times. In addition, existing treatments do not provide adequate relief for the majority of patients and only a small percentage are surgically eligible.

### SUMMARY

In Example 1, a medical device comprising an elongate shaft defining a central channel extending from a proximal end to a distal end of the shaft, the shaft defining a port in communication with the central channel, the shaft further comprising a sharpened distal tip for piercing a bone region of the vertebral body, a handle disposed at the shaft proximal end, an channeling member disposed within the central channel of the shaft, the channeling member defining a proximal end and a curvable distal end, wherein the distal end of the channeling member is movable between a first position, at which the distal end of the channeling member is disposed within the central channel proximal of the port and a second position, at which the distal end of the channeling member is disposed exterior of the central channel and the port, wherein the elongate channeling member is configured to channel through cancellous bone in the vertebral body, a tracking device disposed within the central channel of the shaft and surrounding at least a portion of the channeling member, the tracking device defining a proximal end and a distal end, wherein the distal end of the tracking device is movable between a first position, at which the distal end of the tracking device is disposed within the central channel proximal of the port and a second position, at which the distal end of the tracking device is disposed exterior of the central channel and the port, and a first electrode carried by one of the channeling member and the tracking device, wherein the first electrode is configured to deliver radiofrequency energy to a treatment location when the channeling member and the cannula are both in their respective second positions.

In Example 2, the medical device of Example 1, wherein the medical device is configured for treating a basivertebral nerve in a vertebral body of a patient, and the distal end of the channeling member is configured to channel through cancellous bone in the vertebral body.

In Example 3, the medical device of Examples 1 and/or 2, wherein the port is proximate the distal end and comprises a lateral opening.

In Example 4, the medical device of any of Examples 1-4, wherein the first electrode is coupled with the distal end of the channeling member.

In Example 5, the medical device of any of Examples 1-5, further comprising a second electrode carried by one of the channeling member and the tracking device, wherein the first electrode and the second electrode are configured to deliver bipolar radiofrequency energy to a treatment location when the channeling member and the cannula are both in the second position.

In Example 6, the medical device of any of Examples 1-4 and Example 5, wherein the second electrode comprises a conductive portion of a shaft of the tracking device.

In Example 7, the medical device of any of Examples 1-5 and Example 6, wherein the first electrode is coupled with a distal end of the tracking device.

In Example 8, the medical device of any of Examples 1-7, wherein the shaft is sized for passage through a central channel of an introducer.

In Example 9, the medical device of any of Examples 1-8, wherein the sharpened distal tip of the shaft is configured to pierce cortical bone of the vertebral body and cancellous bone of the vertebral body.

In Example 10, the medical device of any of Examples 1-9, wherein the distal end of the channeling member comprises a sharpened distal tip for defining a path through cancellous bone of the vertebral body.

In Example 11, the medical device of any of Examples 1-10, wherein the curvable distal end of the channeling member has a pre-formed curve.

In Example 12, the medical device of any of Examples 1-11, wherein the port defines a ramp at its distal extremity, the ramp comprising a surface disposed at an angle relative to the central axis of the elongate shaft.

In Example 13, The medical device of any of Examples 1-12, wherein the handle comprises a first actuator, a proximal end of the channeling member is operably connected with the first actuator, wherein the distal end of the channeling member is movable, upon actuation of the first actuator, between the first position and the second position.

In Example 14, the medical device of any of Examples 1-13, wherein the channeling member is coupled with the first actuator via a rack and pinion mechanism.

In Example 15, the medical device of any of Examples 1-14, wherein the handle further comprises a second actuator, wherein the proximal end of the tracking device is operably connected with the second actuator and the tracking device moves upon actuation of the second actuator.

In Example 16, a medical device for treating a basivertebral nerve in a vertebral body of a patient, the medical device comprising an elongate shaft defining a central channel extending from a proximal end to a distal end of the shaft, the shaft defining a port proximate the distal end, the port comprising a lateral opening in communication with the central channel, the shaft further comprising a sharpened distal tip for piercing a bone region of the vertebral body, a handle disposed at the shaft proximal end, the handle comprising a first actuator, an channeling member disposed within the central channel of the shaft, the channeling member defining a proximal end operably connected with the first actuator and a curvable distal end, wherein the distal end of the channeling member is movable, upon actuation of the first actuator, between a first position, at which the distal end of the channeling member is disposed within the central channel proximal of the port and a second position, at which the distal end of the channeling member is disposed exterior of the central channel and the port, wherein the distal end of the channeling member is configured to channel through cancellous bone in the vertebral body, wherein the elongate channeling member is configured to channel through cancellous bone in the vertebral body, a tracking device disposed within the central channel of the shaft and surrounding at least a portion of the channeling member, the tracking device defining a proximal end and a distal end, wherein the distal end of the tracking device is movable between a first position, at which the distal end of the tracking device is disposed within the central channel proximal of the port and a second position, at which the distal end of the tracking device is disposed exterior of the central channel and the port, a first electrode carried by one of the channeling member and the tracking device, and a second electrode carried by one of the channeling member and the tracking device, wherein the first electrode and the second electrode are configured to deliver bipolar radiofrequency energy to a treatment location when the channeling member and the cannula are both in the second position.

In Example 17, the medical device of Example 16, wherein the first electrode is coupled with the distal end of the channeling member.

In Example 18, the medical device of Example 16, wherein the second electrode comprises a conductive portion of a shaft of the tracking device.

In Example 19, the medical device of Example 18, wherein the first electrode is coupled with a distal end of the tracking device.

In Example 20, the medical device of Example 16, wherein the shaft is sized for passage through a central channel of an introducer.

In Example 21, the medical device of Example 16, wherein the sharpened distal tip of the shaft is configured to pierce cortical bone of the vertebral body and cancellous bone of the vertebral body.

In Example 22, the medical device of Example 16, wherein the distal end of the channeling member comprises a sharpened distal tip for defining a path through cancellous bone of the vertebral body.

In Example 23, the medical device of Example 16, wherein the curvable distal end of the channeling member has a pre-formed curve.

In Example 24, the medical device of Example 16, wherein the port defines a ramp at its distal extremity, the ramp comprising a surface disposed at an angle relative to the central axis of the elongate shaft.

In Example 25, the medical device of Example 16, wherein the channeling member is coupled with the first actuator via a rack and pinion mechanism.

In Example 26, the medical device of Example 16, wherein the handle further comprises a second actuator, wherein the proximal end of the tracking device is operably connected with the second actuator and the tracking device moves upon actuation of the second actuator.

In Example 27, a method of treating a basivertebral nerve in a vertebral body of a patient, the method comprising providing a medical device comprising an elongate shaft defining a central channel extending from a proximal end to a distal end of the shaft, the shaft defining a port proximate the distal end, the port comprising a lateral opening in communication with the central channel, the shaft further comprising a sharp distal tip, a handle disposed at the shaft proximal end, the handle comprising a first actuator, an channeling member disposed within the central channel of the shaft, the channeling member operably connected with the first actuator and defining a distal end, a tracking device disposed within the central channel of the shaft and surrounding at least a portion of the channeling member, the tracking device defining a distal end. a pair of electrodes, each electrode of the pair electrodes respectively carried by one of the channeling member and the tracking device; actuating the first actuator to advance the distal end of the channeling member out of the port to channel within a cancellous bone region of the vertebral body to a treatment location proximate the basivertebral nerve via the pair of electrodes, applying radiofrequency energy to the treatment location to modulate the basivertebral nerve.

In Example 28, the method of Example 27, wherein the channeling member has a distal section with a preformed curve.

In Example 29, the method of Example 27, wherein the handle defines a proximal striking surface, and the method further comprises striking the striking surface with a mallet to advance the distal tip of the shaft through cortical bone in the vertebral body and into the cancellous bone region of the vertebral body.

In Example 30, the method of Example 29, further comprising inserting the shaft of the medical device through an introducer such that the sharp distal tip protrudes from an axial opening at a distal end of the introducer.

In Example 31, the method of Example 27, wherein the handle further comprises a second actuator and the tracking device is operably connected with the second actuator; and the method further comprises actuating the second actuator to advance tracking device over the channeling member to the treatment location.

In Example 32, a method of treating a basivertebral nerve in a vertebral body of a patient, the method comprising providing a medical device comprising a cannula defining a central channel extending from a proximal end to a distal end of the cannula, the cannula defining a lateral port proximate the distal end and in communication with the central channel, the cannula further comprising a sharp distal tip, a handle disposed at the cannula proximal end, the handle comprising a first actuator, a stylet disposed within the central channel of the cannula, the stylet defining a proximal end and a distal end, wherein the stylet is coupled with the first actuator and axially movable, in response to actuation of the first actuator, such that the distal end of the stylet is advanceable out of the lateral port and retractable into the central channel of the cannula, and a cannulated probe disposed within the central channel of the shaft and surrounding at least a portion of the stylet, the cannulated probe defining a proximal end and a distal end, the cannulated probe further comprising a pair of electrodes, wherein the cannulated probe axially movable, in response to actuation of the second actuator, such that the distal end of the cannulated probe is advanceable out of the lateral port and retractable into the central channel of the cannula, inserting the cannula of the medical device through cortical bone of the vertebral body into a cancellous bone region of the vertebral body, actuating the first actuator to advance the distal end of the stylet out of the lateral port and through the cancellous bone region to a treatment location proximate the basivertebral nerve, via the pair of electrodes, applying radiofrequency energy to the treatment location to treat the basivertebral nerve.

In Example 33, the method of Example 32, wherein at least the distal end of the stylet has a preformed curvature such that, as the stylet is advanced out of the lateral port defined in the shaft of the medical device, the stylet curves away from a central axis of the shaft of the medical device.

In Example 34, the method of Example 32, wherein the operation of actuating the first actuator comprises rotating a thumbwheel.

In Example 35, the method of Example 32, wherein the handle further comprises a second actuator and the cannulated probe is coupled with the second actuator, the method further comprises actuating the second actuator to advance the distal end of the cannulated probe over the channeling member out of the lateral port to the treatment location.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic illustration of vertebral levels and vertebrae that can be treated by systems and methods consistent with various aspects of the present disclosure.

FIG. 2 is a side view of a medical device, consistent with various aspects of the present disclosure.

FIGS. 3A-3H are close-up views of the medical device as indicated by the phantom circle 3 in FIG. 2. More specifically, FIG. 3A is a close-up, cross-sectional view of the introducer, consistent with various aspects of the present disclosure. FIG. 3B is a top view of an alternative tip of the introducer, consistent with various aspects of the present disclosure.

FIG. 3C is a side view of the alternative tip of the introducer, consistent with various aspects of the present disclosure. FIG. 3D is a side view of a port of the introducer, consistent with various aspects of the present disclosure. FIG. 3E is a side view of an alternative port of the introducer, consistent with various aspects of the present disclosure. FIG. 3F is a close-up, top view of the tracking device, consistent with various aspects of the present disclosure. FIG. 3G is a close-up, cross-sectional view of the channeling device, consistent with various aspects of the present disclosure. FIG. 3H is a close-up, top view of an alternative tracking device, consistent with various aspects of the present disclosure.

FIG. 4 is a rear view of the medical device, consistent with various aspects of the present disclosure.

FIG. 5 is a cross-sectional view of a handle of the medical device as indicated by line 5-5 in FIG. 4, consistent with various aspects of the present disclosure.

FIG. 6 is a flowchart of a method of accessing and treating tissue within a vertebral body, consistent with various aspects of the present disclosure.

FIGS. 7A-7D are a series of top views of operations of accessing and treating tissue within a vertebral body, consistent with various aspects of the present disclosure.

FIG. 8A is a cross-sectional view of an alternative handle of the medical device with the same orientation as that of FIG. 5, wherein the handle is in a retracted configuration, consistent with various aspects of the present disclosure.

FIG. 8B is a cross-sectional view of an alternative handle of the medical device with the same orientation as that of FIG. 5, wherein the handle is in an extended configuration, consistent with various aspects of the present disclosure.

FIG. 9 is a cross-sectional view of a steerable tip, consistent with various aspects of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

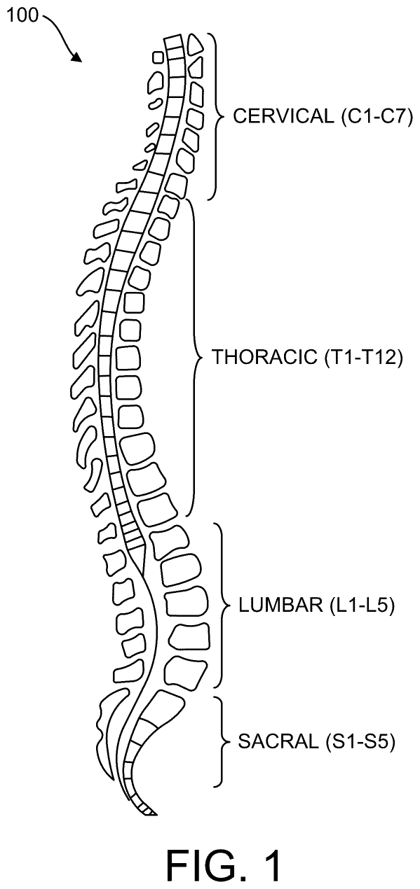

FIG. 1 is a schematic illustration of a human spine 100 including vertebral levels and vertebrae that can be treated by systems and methods consistent with various aspects of the present disclosure. Treatment procedures may include modulation of nerves within or surrounding bones. The terms “modulation” or “neuromodulation”, as used herein, shall be given their ordinary meaning and shall also include ablation, permanent denervation, temporary denervation, disruption, blocking, inhibition, electroporation, therapeutic stimulation, diagnostic stimulation, inhibition, necrosis, desensitization, or other effect on tissue. Neuromodulation shall refer to modulation of a nerve (structurally and/or functionally) and/or neurotransmission. Modulation is not necessarily limited to nerves and may include effects on other tissue, such as tumors or other soft tissue.

Embodiments described herein are directed to systems and methods for modulating nerves within or adjacent (e.g., surrounding) bone. In some embodiments, an intraosseous nerve (e.g., basivertebral nerve (BVN)) within a vertebral body of the spine is modulated for treatment or prevention of chronic back pain. The vertebral body may be located in any level of the vertebral column (e.g., cervical, thoracic, lumbar and/or sacral). Multiple vertebral bodies may be treated in a single visit or procedure (simultaneously or sequentially). The multiple vertebral bodies may be located in a single spine segment or in different spine segments. Examples of the former are two adjacent vertebral bodies in the sacral spine segment (e.g., S1 and S2) or lumbar spine segment (e.g., L3, L4 and/or L5) or thoracic spine segment or cervical spine segment), and an example of the latter is an L5 vertebra in the lumbar spine segment and an S1 vertebra in the sacral spine segment. Intraosseous nerves within bones other than vertebral bodies may also be modulated. For example, nerves within a humerus, radius, femur, tibia, calcaneus, tarsal bones, hips, knees, and/or phalanges can be modulated.

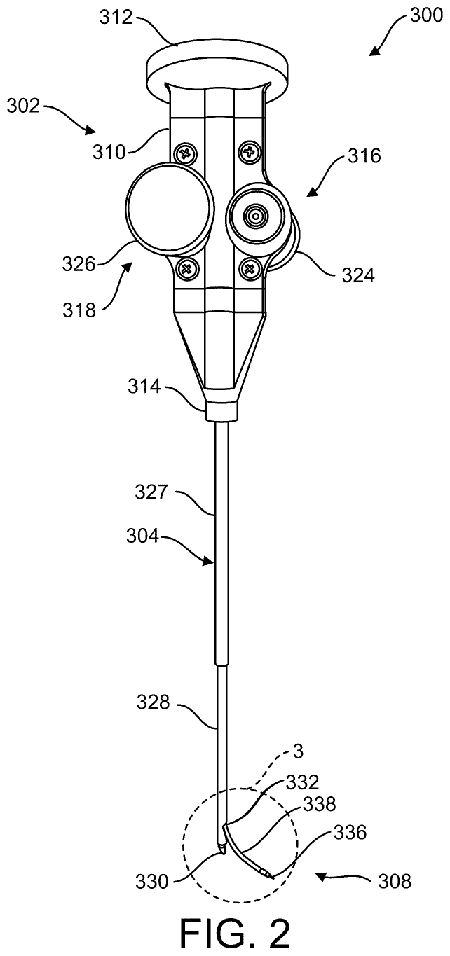

FIG. 2 is shows a medical device 300 for accessing a vertebral body and delivering radiofrequency energy. As shown in FIG. 2, the medical device 300 includes a handle 302, an introducer shaft 304, and extendable therapy components 308. The medical device 300 is configured to access the vertebral body, position therapy components adjacent the treatment tissue (e.g., the basivertebral nerve) and deliver sufficient radiofrequency energy to the nerve to modulate (e.g., ablate) the nerve so as to reduce or eliminate a patient's pain.

As shown, the handle 302 includes a body 310, including a proximal end 312 and a distal coupling feature 314, and it further includes a first actuator 316 and a second actuator 318. In the exemplary embodiment of FIG. 2, the proximal end 312 is substantially disc-shaped and has sufficient strength to serve as a “malletable” surface. In other words, during use, the physician may strike the proximal end 312 with a rubber mallet with sufficient force to drive the distal end of the introducer shaft 304 through the cortical bone of the vertebral body. The handle further includes a coupling feature 314 to engage with the introducer shaft 304. The actuators 316 and 318 and coupled to the extendable therapy components 308 and operate to drive these components out of the introducer shaft 304 and toward the basivertebral nerve. Each of the actuators 316 and 318 include corresponding knobs 324 and 326, which are configured to be readily manipulated (e.g., rotated) by the physician.

As shown in FIG. 2, the introducer shaft 304 extends distally from the coupling feature 314 of the handle 302 in a longitudinal direction. The introducer shaft 304 has a thicker proximal section 327 joined to a thinner distal section 328 that terminates in a penetrating tip 330. A proximal portion of the proximal section 327 is disposed inside and coupled to the handle 302. The introducer shaft 304 includes a bore or lumen, which extends from the proximal end to a lateral or radial opening (or port) 332, which is sized and shaped to allow the extendable therapy components 308 to exit therethrough. In some embodiments, the proximal section 327 has a diameter between about 4.7 millimeters (mm) to about 6.3 mm, while the distal section 328 has a diameter between about 2.5 mm to about 4.4 mm. In some embodiments, the diameter of the distal section 328 is 11 gauge (i.e., between about 2.97 and 3.07 mm).

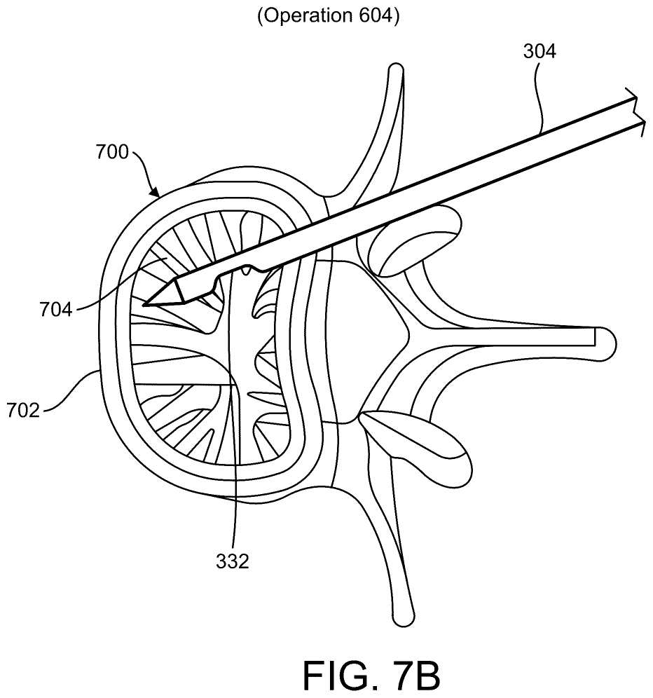

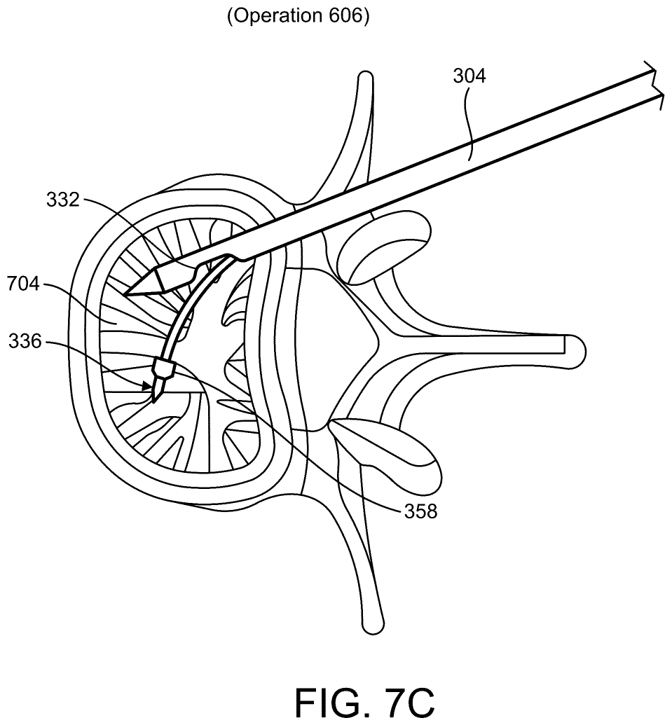

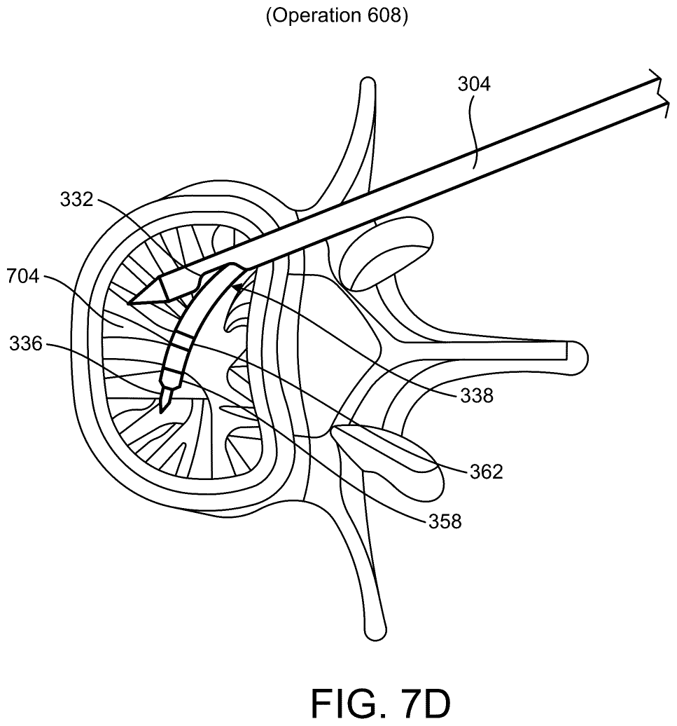

The extendable therapy components 308 include proximal portions disposed inside the handle 302 that are coupled to the actuators 316, 318, respectively. These proximal portions extend distally through the bore in the shaft to a location near the lateral opening 332. As shown in FIG. 2, the extendable therapy components 308 are in their extended positions, i.e., the physician has manipulated the actuators 316, 318 an amount sufficient to advance the extendable therapy components 308 out of the introducer shaft 304 through the lateral opening 332. When the physician has manipulated the actuators 316, 318 to retract the extendable therapy components 308, the extendable therapy components 308 may be fully retracted (through the lateral opening 332) such that they are fully nested within the bore of the shaft 304 (as shown in FIGS. 7A and 7B).

As shown in FIG. 2, the extendable therapy components 308 include a channeling device (e.g., a guidewire) 336 and a tracking device 338 (e.g., a hypotube). Each of the channeling device 336 and tracking device 338 are coupled at a proximal end to one of the actuators 316, 318, respectively, and extend through the bore in the introducer shaft 304. During use, the physician can independently manipulate each of these components 336, 338 to cause them to extend from the introducer shaft 304 toward the basivertebral nerve and to retract the components 336, 338 into the shaft 304.

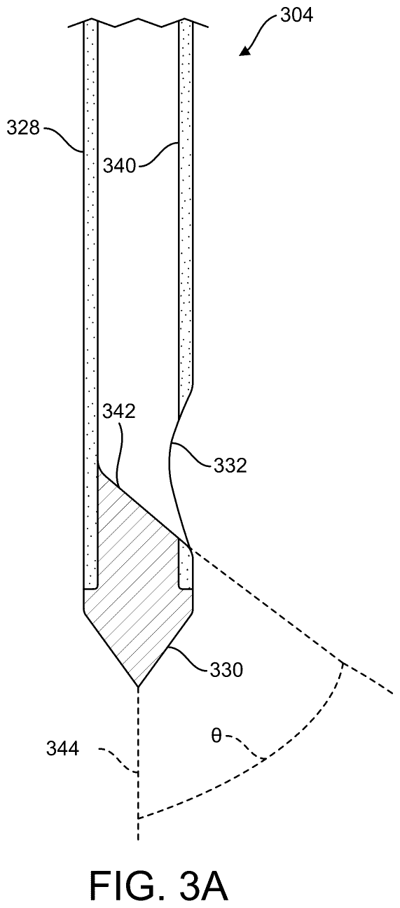

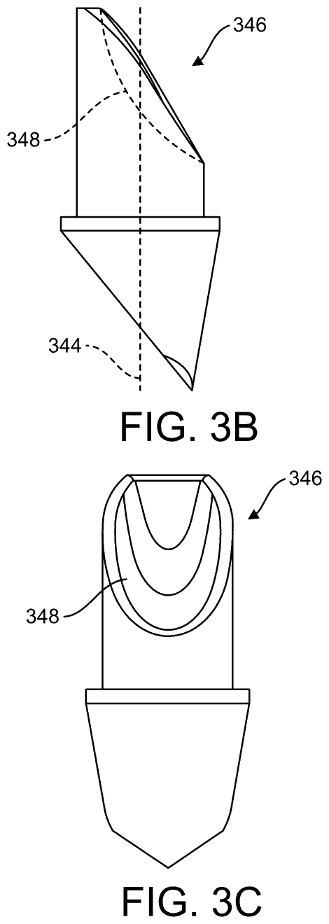

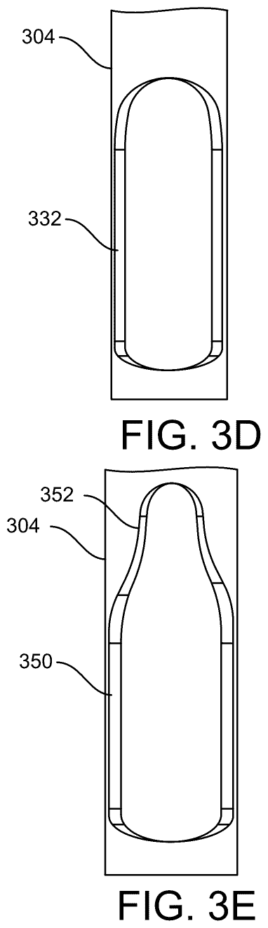

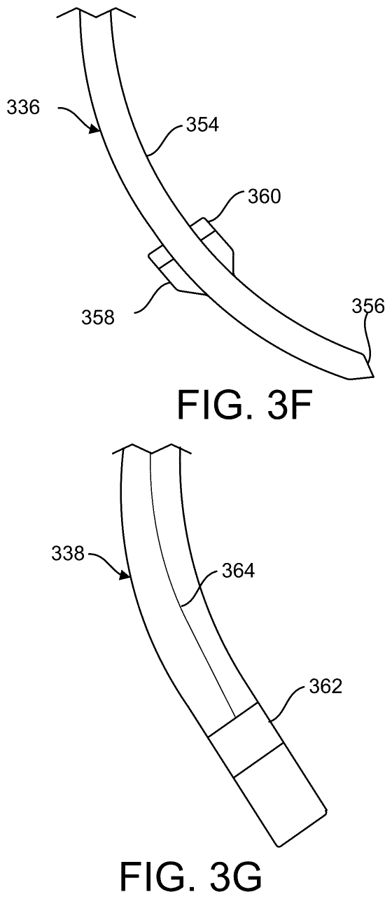

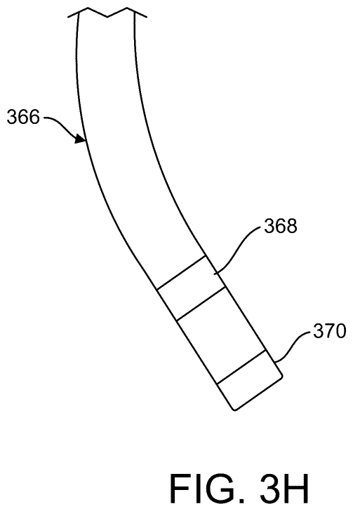

FIGS. 3A-3E show detailed views of various aspects of the distal portion of the introducer shaft 304 indicated by the phantom circle 3 in FIG. 2. FIG. 3A is a cross-sectional view of the introducer shaft 304. The introducer shaft 304 includes an inner surface 340 defining the longitudinally extending bore and is breached at the distal end by the lateral opening 332. The tip 330 is coupled to the distal end of the introducer shaft 304 and defines a ramp surface 342. The tip 330 is coupled to the introducer shaft 304 such that the ramp surface 342 is circumferentially opposed to the port 332. The ramp 342 that is at an angle θ with respect to the central axis 344 of the introducer shaft 304. In some embodiments, angle θ is between thirty and sixty degrees. This configuration also allows for the sharpened tip 330 occupy the entire cross-sectional footprint of the distal end of the introducer shaft 304. In some embodiments, the tip 330 is replaceable (e.g., by being threaded into the distal section 328) such that the physician can replace a worn tip and/or choose a different style/shape of tip. However, in other embodiments, the tip can be integral to the introducer shaft, so that the ramp surface would be defined by the inner wall.

FIG. 3B is a top view of an alternative tip 346 of the introducer shaft 304. In the illustrated embodiment, in contrast with the tip 330 (shown in FIG. 3A), the tip 346 is asymmetric with respect to the central axis 344. More specifically, the very distal end of tip 346 is biased towards the inside of the curvature that the channeling device 336 makes when extended into cancellous bone 704.

FIG. 3C is a side view of the alternative tip 346 of the introducer shaft 304. In the illustrated embodiment, in contrast with the ramp 342 (shown in FIG. 3A), the ramp 348 has a concave shape. While the proximal-to-distal overall angle of the ramp 348 may be the same as that of the ramp 342, the local angles on the ramp 348 can deviate therefrom.

FIG. 3D is a side view of the lateral opening 332, which is a lateral or radial opening through the wall of the introducer shaft 304. In a side projection of the lateral opening 332, the lateral opening 332 has an oval shape that can result from, for example, a rotating vertical mill bit cutting in from the side of the introducer shaft 304 towards the center axis 344 (shown in FIG. 3A).

FIG. 3E is a side view of an alternative lateral opening 350, which is a lateral opening through the wall of the introducer shaft 304. In a side projection of the lateral opening 350, the lateral opening 350 has a bottle shape with a narrower neck region 352 at the proximal end of the lateral opening 350. The vertical width of the neck region 352 (which is shown extending horizontally given the orientation of FIG. 3E) is still wider than the channeling device 336, so the channeling device 336 has less room to deviate vertically (left-and-right, as shown in FIG. 3E). Thus, the neck region 352 is configured to better guide the channeling device 336 as it exits from introducer shaft 304, which reduces the chance and/or amount of vertical deviation of the channeling device 336 in the vertebral body.

FIG. 3F is a close-up, cross-sectional view of the channeling device 336 as indicated by the phantom circle 3 in FIG. 2. In the illustrated embodiment, the channeling device 336 is a curved elongate member (e.g., a shaft or wire) 354. As shown, the shaft 354 has a sharpened tip 356 at the distal end of the shaft 354. However, in some embodiments, the distal end of the channeling device 336 has a pre-formed curve (e.g., to the right, as shown in FIG. 3C) when extended (i.e., when unconfined) but can elastically deform enough to fit into the introducer shaft 304 and the tracking device 338 when retracted. In addition, in some embodiments, the channeling device 336 includes the bulb 358 that is welded to the shaft 354 at a position proximal from the tip 356. For example, the bulb 358 can be five to fifteen diameters of the channeling device 336 from the tip 356. In addition, an electrical insulator 360 is be positioned on the channeling device 336, on the proximal side of the bulb 358. The insulator 360 electrically insulates the channeling device 336 and the bulb 358 from the tracking device 338 (shown in FIG. 3G), for example, during treatment. In various embodiments, the bulb 358, which is electrically coupled to the shaft or wire 354, is configured to function as an electrode.

In some embodiments, the diameter of the shaft 354 is between about 1.0 mm to about 1.8 mm, and in some embodiments, the diameter of the shaft 354 is about 1.4 mm. In some embodiments, the diameter of the bulb 358 is between about 2.0 mm to about 2.8 mm, and in some embodiments, the diameter of the bulb 358 is about 2.3 mm. In some embodiments, the shaft 354 comprises nickel titanium (nitinol), stainless steel, and/or polyether ether ketone (PEEK) materials. In some embodiments, the bulb 358 comprises stainless steel, nitinol, titanium, PEEK, (glass filled) nylon polymer, and/or (glass filled) polycarbonate materials. In embodiments where the bulb 358 functions as an electrode, an electrically conductive material will comprise at least a portion of the bulb 358. In some embodiments, the insulator 360 comprises PEEK, fluorinated ethylene propylene (FEB), polyether block amide (PEBA), nylon, polyethylene terephthalate (PET), polyvinyl chloride (PVC), and/or polytetrafluoroethylene (PTFE) materials.

FIG. 3G is a schematic view of the tracking device 338 as indicated by the phantom circle 3 in FIG. 2. In the illustrated embodiment, the tracking device 338 is a curved, hollow tubular member. However, in some embodiments, the distal end of the tracking device 338 has a pre-formed curve (e.g., to the right, as shown in FIG. 3G) when extended (i.e., when unconfined) but can elastically deform enough to fit into the introducer shaft 304 when retracted. The tracking device 338 is sized and shaped to fit over and slide along (i.e., follow or track) the shaft of the channeling device. In some embodiments, the tracking device 338 comprises nickel titanium (nitinol), stainless steel, and/or polyether ether ketone (PEEK) materials.

As shown in FIG. 3G, the tracking device 338 includes an electrode 362 near the distal end that is electrically connected to an electrical connector (not shown) on the handle 302. In certain embodiments, the tracking device is electrically conductive (e.g., a metallic hypotube) and the tracking device can then function to deliver electrical energy along the length of the device to the electrode 362. In other embodiments, the tracking device is not conductive (e.g., a catheter), the tracking device includes a conductor 364 electrically coupled to the electrode and extending to the electrical connector on the proximal handle. The electrode 362 works alone in a monopolar treatment arrangement or in conjunction with the channeling device 336 (shown in FIG. 3F) in a bipolar treatment arrangement (i.e., the bulb 358 acts as the second electrode). In such bipolar embodiments, the shaft 354 (shown in FIG. 3F) is electrified during treatment, so the interior of the tracking device 338 includes an electrically insulating sheath (not shown).

FIG. 3H is a schematic view of an alternative tracking device 366. In the illustrated embodiment, the tracking device 366 is a cannulated probe that includes two electrodes 368, 370-a ring electrode 368 near the distal end and a tip electrode 370. The electrodes 368, 370 are electrically insulated from each other and are electrically coupled to the electrical connector, for example, by independent leads (not shown) and/or using the body of the tracking device 338. In such embodiments, the channeling device 336 (shown in FIG. 3F) is electrically insulated from the tracking device 336 and is not used to deliver therapeutic energy to tissue.

Further alternative embodiments of the tracking devices 338, 366 and the channeling device 336 are shown in FIGS. 3B, 3C, and 3H, respectively. For example, in some alternative embodiments, the channeling device 336 and the tracking device 338, 366 are straight instead of having a preset curvature In such embodiments, the ramp 342 (shown in FIG. 3A) is oriented and positioned such that when the channeling device 336 is advanced through the introducer shaft 304, the channeling device 336 will contact the ramp 342 and be forced out through the lateral opening 332.

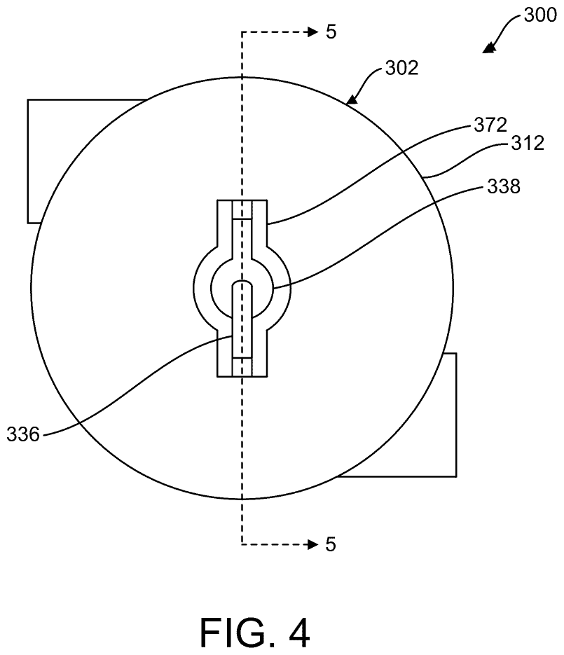

FIG. 4 shows the proximal end of the handle of the medical device 300. In the illustrated embodiment, the strikable proximal end 312 includes a double keyway slot 372 in which the proximal ends of the channeling device 336 and the tracking device 338 are slidably positioned (the gap between the channeling device 336/tracking device 338 and the slot 372 has been exaggerated for illustrative purposes). While the slot 372 extends through the handle 302 in some embodiments, in other embodiments, the slot 372 is blind and does not extend through to the proximal side of the strikable end 312. In addition, the channeling device 336 is positioned on the central axis 344 (shown in FIG. 3A) and the proximal end of the tracking device 338 has a U-shaped configuration that only partially surrounds the proximal end of the channeling device 336. In various embodiments, the handle further includes an electrical connection (not shown) coupled to each of the channeling device 336 and the tracking device 338. Such an electrical connection allows each of these devices to be electrically coupled to a radiofrequency generator to supply energy for the therapy.

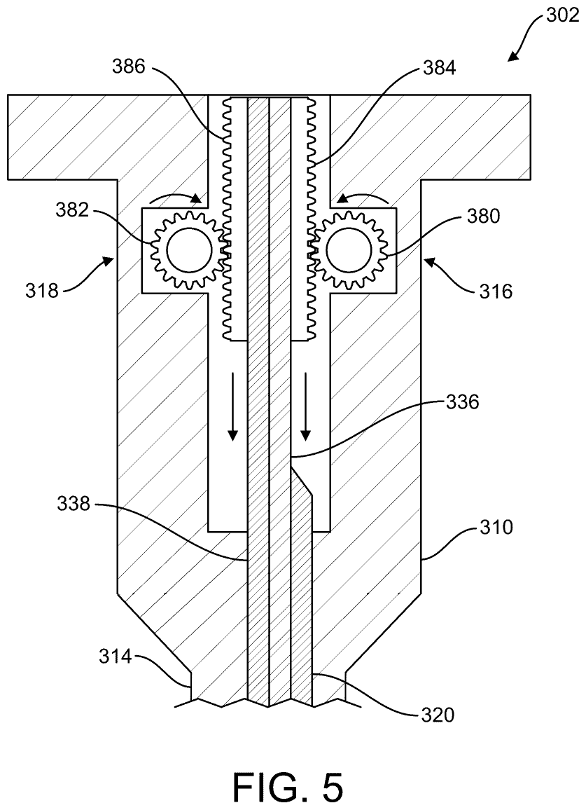

FIG. 5 is a cross-sectional view of the handle 302 as indicated by line 5-5 in FIG. 4 and shows an exemplary actuation mechanism for extending the retracting the channeling device 336 and the tracking device 338. In the illustrated embodiment, actuators 316, 318 include pinion gears 380, 382 which are connected to knobs 324, 326 (shown in FIG. 2), respectively. The actuators 316, 318 further include racks 384, 386 that are connected to the proximal ends of the channeling device 336 and the tracking device 338, respectively. Thereby, the physician can turn the knob 324 in the indicated direction (i.e., counterclockwise) to advance the channeling device 336 (downward, as indicated in FIG. 5), and the operator can turn the knob 326 in the indicated direction (i.e., clockwise) to advance the tracking device 338 (downward, as indicated in FIG. 5). Furthermore, turning the knobs 324, 326 oppositely will retract the channeling device 336 and the tracking device 338, respectively. As further shown, the tracking device 338 transitions from partially surrounding to completely surrounding the channeling device 336 prior to exiting the coupling feature 314.

In some embodiments, the tracking device 338 and the channeling device 336 are extended simultaneously. Similarly, in some such embodiments, the tracking device 338 and the channeling device 336 are retracted simultaneously. Therefore, in some such embodiments, the handle 302 would only include a single actuator with a single knob that would be functionally connected to actuate both the channeling device and the tracking device simultaneously.

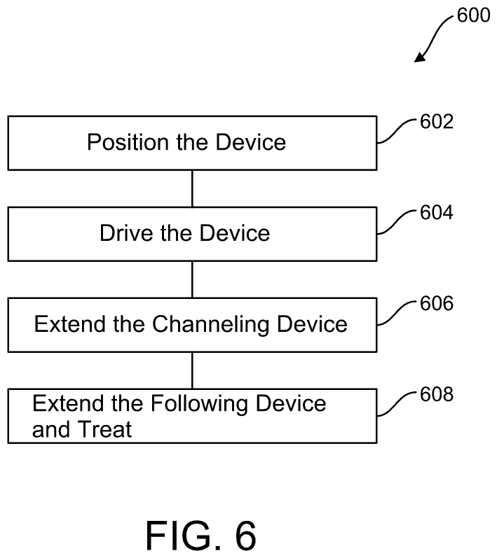

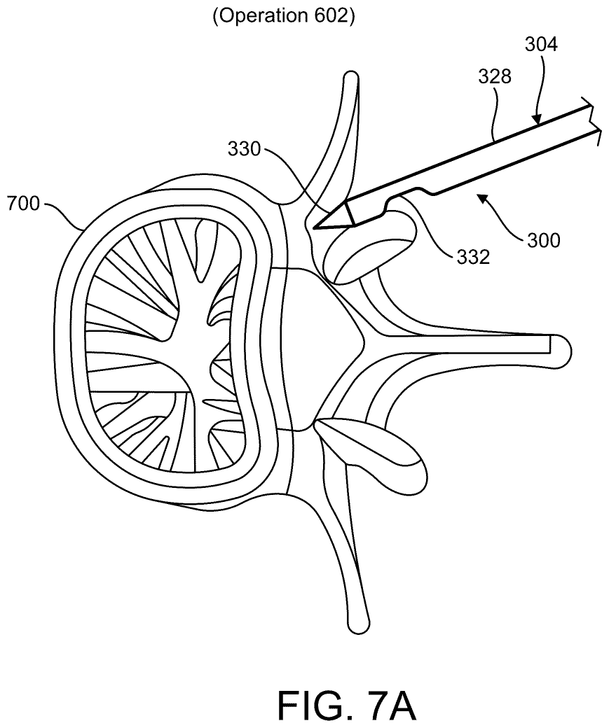

FIG. 6 is a flowchart illustrating a method 600 of accessing and treating tissue (e.g., a basivertebral nerve) within the vertebral body 700 using the medical device 300. As shown, the method 600 includes positioning the medical device 300 (block 602), driving the introducer shaft 304 through the cortical bone (block 604), extending the channeling device 336 (block 606) and extending the tracking device 338 and delivering therapy (block 608). FIGS. 7A-7D are a series of top views of operations 602-608 of accessing and treating tissue within the vertebral body 700 using the medical device 300. FIGS. 2 and 3A-3D will now be discussed in conjunction with one another, and each operation of the method 600 is illustrated by a corresponding one of FIGS. 7A-7D.

In the illustrated embodiment, the method 600 begins at the operation 602, wherein the medical device 300 is positioned against the vertebral body 700. The device 300 includes an introducer shaft 304 with a sharpened tip 330 and a hollow shaft 304. The device 300 also includes a lateral opening 332 positioned near the tip 330 that connects with the interior cavity of the introducer shaft 304.

At the operation 604, the introducer shaft 304 is driven through the cortical bone region 702 and into the cancellous bone region 704 a sufficient distance such that the lateral opening 332 is located at least in part in the cancellous bone region 704.

At the operation 606, a channeling device 336 is extended into the cancellous bone region 704 from the interior of the introducer shaft 304 through the lateral opening 332.

At operation 608, a tracking device 338 is extended into the cancellous bone region 704 from the interior of the introducer shaft 304 through the lateral opening 332. The tracking device 338 is a hollow elongate member that surrounds the channeling device 336 and can be extended along the channeling device 336 until the distal end of the tracking device 338 contacts the bulb 358 which is larger in outer diameter than the outer diameter of the tracking device 338. Thus, the bulb 358 can clear a path through the cancellous bone region 704 for the tracking device 338 to follow. The tracking device 338 includes the electrode 362 at or near the distal end that is electrically insulated from the channeling device 336. Furthermore, additional electrical insulation lines the interior of the tracking device 338, for example, in embodiments where the entirety of the channeling device 336 is electrically conductive and the channeling device 336 functions as a stylet. Once the distal tip of the channeling device 336 and the electrode 362 of the tracking device 338 are properly positioned, for example, at or near the BVN arborization point and/or 1-2 mm anterior of the coronal/frontal midplane of the vertebral body, treatment can commence by passing radiofrequency electrical energy from a radiofrequency generator (not shown) through the channeling device 336 to the electrode 362 to ablate the BVN.

One embodiment of the medical device 300 is shown in FIGS. 7A-7D, to which there can be alternatives. For example, in some embodiments, operations 206 and 208 occur simultaneously (i.e., the tracking device 338 is extended along with the channeling device 336). In certain embodiments, the tracking device 338 is mechanically coupled to the channeling device 336, such that they necessarily extend simultaneously.

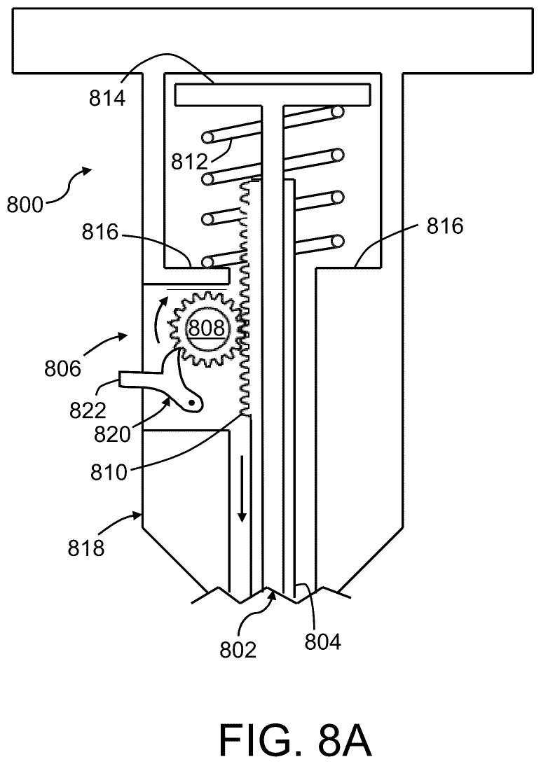

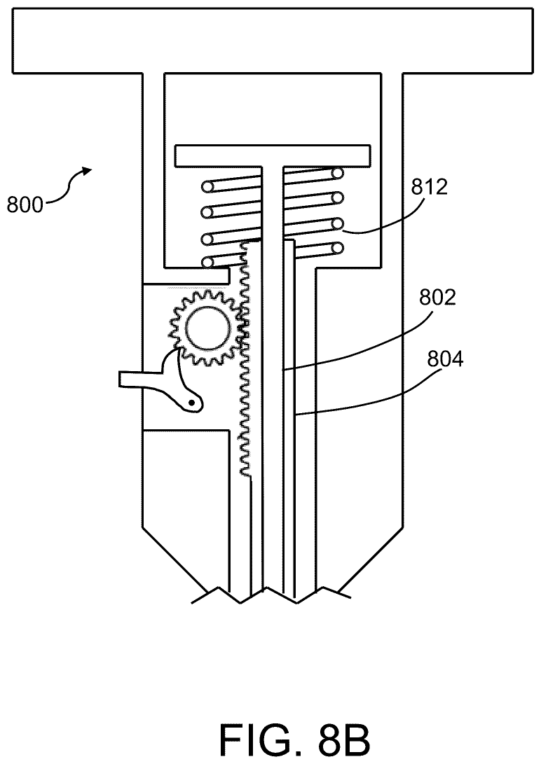

FIG. 8A is a cross-sectional view of an alternative handle 800 in a retracted configuration. In the illustrated embodiment, handle 800 is an exemplary actuation mechanism for extending and retracting a channeling device 802 and a tracking device 804 simultaneously. Therefore, there is only a single actuator 806 that includes a pinion gear 808 which is connected to a knob (not shown). The actuator 806 further includes a rack 810 that is connected to the proximal end of the tracking device 804. Thereby, the physician can turn the knob in the indicated direction (i.e., counterclockwise) to advance the devices 802, 804 (downward, as indicated in FIG. 8A).

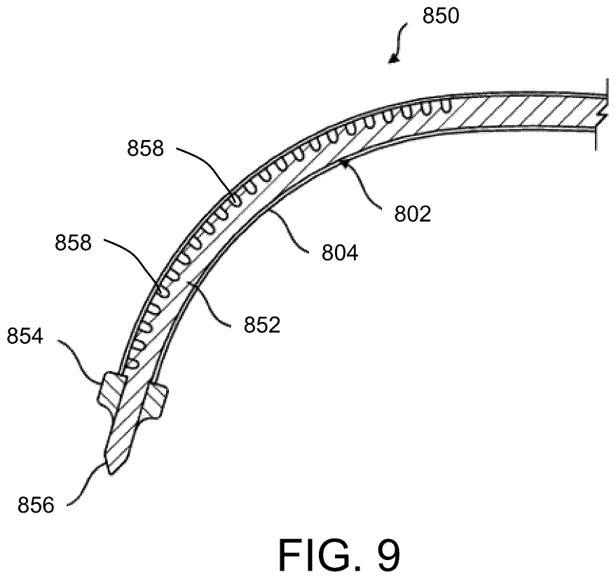

In the illustrated embodiment, the handle 800 includes a biasing member 812 (e.g., a spring) that exerts a proximal force on the channeling device 802 that urges the proximal end of the channeling device 802 away from the proximal end of the tracking device 804 (upward, as shown in FIG. 8A). As shown in FIG. 9, the devices 802, 804 move together because the distal ends of the devices 802, 804 are engaging each other. In some embodiments, the biasing member 812 is a coil spring that is positioned between a cap 814 of the channeling device 802 and a shelf 816 of the handle body 818. The resultant force from the biasing member 812 is in opposition to the direction of advancement of the devices 802, 804. Thus, the handle 800 includes a pawl 820 that prevents the pinion gear 808 from reversing (i.e., rotating clockwise). However, when the physician desires to retract the devices 802, 804, the physician can disengage the pawl 820 by exerting force on an extension 822. Then, the pawl 820 will rotate away from the pinion gear 808, disengaging the two components and allowing the pinion gear 808 to reverse.

FIG. 8B is a cross-sectional view of the alternative handle 800 in an extended configuration. In the illustrated embodiment, the physician has extended the devices 802, 804 which compresses the biasing member 812. Thereby, the amount of force exerted by the biasing member 812 against the channeling device 802 has increased.

FIG. 9 is a cross-sectional view of a steerable tip 850. In the illustrated embodiment, the tip 850 includes the channeling device 802 and the tracking device 804 that surrounds most of the channeling device 802. The channeling device 802 includes a wire 852 positioned mostly inside of the tracking device 804 and a bulb 854 connected (e.g., welded) to a thinned portion of the wire 852. The bulb 854 is located proximal to a sharpened end 856 of the wire 852 (e.g., between one and five diameters of the bulb 854). The arrangement of the wire 852 extending from the bulb 854 forms a narwhal tip (i.e., a tip with a smaller diameter segment adjacent to a larger diameter segment that is proximal therefrom). The channeling device 802 slidably receives the tracking device 804, but the outer diameter of the bulb 854 is larger than the inner diameter of the tracking device 804. Thereby, when the tracking device 804 is advanced by the actuator 806 (shown in FIG. 8A), the distal end of the tracking device 804 will exert force on the proximal end of the bulb 854, which advances the channeling device 802. Furthermore, the axial force that is applied to the channeling device 802 by the biasing member 812 (shown in FIG. 8B) is resisted by the tracking device 804. Thus, the channeling device 802 is in tension while the tracking device 804 is in compression. The differential in the tension and compression is further increased as the devices 802, 804 are advanced because the force from the biasing member 812 increases upon advancement of the devices 802, 804.

In the illustrated embodiment, the wire 852 includes an array of notches 858 that are transverse cuts that extend part of the way (e.g., halfway) through the wire 852 that make one side of the wire 852 (i.e., the top/left side, as shown in FIG. 9) axially weaker compared to the circumferentially opposite side (i.e., the bottom/right side, as shown in FIG. 9). The weaker side of the channeling device 802 is less able to resist the axial tensioning force exerted by the biasing member 812 compared to the stronger side of the channeling device 802 is. Thus, the channeling device 802 will bend or deflect towards the stronger side (i.e., downwards, as shown in FIG. 9). This bending effect is increased as the tip 850 is advanced due to the increased force from the biasing member 812.

In the illustrated embodiment, the outer diameter of the bulb 854 is larger than the outer diameter of the tracking device 804. Thereby, the bulb 854 can clear a path through vertebral body for the tracking device 804. In addition, the decreased size of the end 856 allows the channeling device 802 to bite into the vertebral body more easily than if the end had a diameter similar to that of the tracking device 804 or the bulb 854. When combined with the bending effect due to the tension in the channeling device 802, the tip 850 can have a tight curvature in the vertebral body.

While one embodiment of the steerable tip 850 is shown in the present disclosure, other embodiments are possible. For examples and disclosures of such embodiments, the patent application Ser. No. 63/676,170 entitled “STEERABLE ASSEMBLY FOR TREATING A VERTEBRAL BODY” by Parker et al. filed on 2024 Jul. 26 is hereby incorporated by reference to the present disclosure.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A medical device for treating a basivertebral nerve in a vertebral body of a patient, the medical device comprising:
an elongate shaft defining a central channel extending from a proximal end to a distal end of the shaft, the shaft defining a port proximate the distal end, the port comprising a lateral opening in communication with the central channel, the shaft further comprising a sharpened distal tip for piercing a bone region of the vertebral body;
a handle disposed at the shaft proximal end, the handle comprising a first actuator;
an channeling member disposed within the central channel of the shaft, the channeling member defining a proximal end operably connected with the first actuator and a curvable distal end, wherein the distal end of the channeling member is movable, upon actuation of the first actuator, between a first position, at which the distal end of the channeling member is disposed within the central channel proximal of the port and a second position, at which the distal end of the channeling member is disposed exterior of the central channel and the port, wherein the distal end of the channeling member is configured to channel through cancellous bone in the vertebral body;
wherein the elongate channeling member is configured to channel through cancellous bone in the vertebral body;
a tracking device disposed within the central channel of the shaft and surrounding at least a portion of the channeling member, the tracking device defining a proximal end and a distal end, wherein the distal end of the tracking device is movable between a first position, at which the distal end of the tracking device is disposed within the central channel proximal of the port and a second position, at which the distal end of the tracking device is disposed exterior of the central channel and the port;
a first electrode carried by one of the channeling member and the tracking device; and
a second electrode carried by one of the channeling member and the tracking device;
wherein the first electrode and the second electrode are configured to deliver bipolar radiofrequency energy to a treatment location when the channeling member and the cannula are both in the second position.

2. The medical device of claim 1, wherein the channeling member is a guidewire and the first electrode is coupled with the distal end of the guidewire.

3. The medical device of claim 1, wherein the tracking device is a hypotube and the second electrode comprises a conductive portion of a shaft of the hypotube.

4. The medical device of claim 3, wherein the first electrode is coupled with a distal end of the hypotube.

5. The medical device of claim 1, wherein the shaft is sized for passage through a central channel of an introducer.

6. The medical device of claim 1, wherein the sharpened distal tip of the shaft is configured to pierce cortical bone of the vertebral body and cancellous bone of the vertebral body.

7. The medical device of claim 1, wherein the distal end of the channeling member comprises a sharpened distal tip for defining a path through cancellous bone of the vertebral body.

8. The medical device of claim 1, wherein the curvable distal end of the channeling member has a pre-formed curve.

9. The medical device of claim 1, wherein the port defines a ramp at its distal extremity, the ramp comprising a surface disposed at an angle relative to the central axis of the elongate shaft.

10. The medical device of claim 1, wherein the channeling member is coupled with the first actuator via a rack and pinion mechanism.

11. The medical device of claim 1, wherein the handle further comprises a second actuator, wherein the proximal end of the tracking device is operably connected with the second actuator and the tracking device moves upon actuation of the second actuator.

12. A method of treating a basivertebral nerve in a vertebral body of a patient, the method comprising:
providing a medical device comprising:
an elongate shaft defining a central channel extending from a proximal end to a distal end of the shaft, the shaft defining a port proximate the distal end, the port comprising a lateral opening in communication with the central channel, the shaft further comprising a sharp distal tip;
a handle disposed at the shaft proximal end, the handle comprising a first actuator;
an channeling member disposed within the central channel of the shaft, the channeling member operably connected with the first actuator and defining a distal end;
a tracking device disposed within the central channel of the shaft and surrounding at least a portion of the channeling member, the tracking device defining a distal end;
a pair of electrodes, each electrode of the pair of electrodes respectively carried by one of the channeling member and the tracking device;
actuating the first actuator to advance the distal end of the channeling member out of the port to channel within a cancellous bone region of the vertebral body to a treatment location proximate the basivertebral nerve; and
via the pair of electrodes, applying radiofrequency energy to the treatment location to modulate the basivertebral nerve.

13. The method of claim 12, wherein the channeling member has a distal section with a preformed curve.

14. The method of claim 12, wherein the handle defines a proximal striking surface, and the method further comprises striking the striking surface with a mallet to advance the distal tip of the shaft through cortical bone in the vertebral body and into the cancellous bone region of the vertebral body.

15. The method of claim 14, further comprising inserting the shaft of the medical device through an introducer such that the sharp distal tip protrudes from an axial opening at a distal end of the introducer.

16. The method of claim 12, wherein:
the handle further comprises a second actuator and the tracking device is operably connected with the second actuator; and
the method further comprises actuating the second actuator to advance tracking device over the channeling member to the treatment location.

17. A method of treating a basivertebral nerve in a vertebral body of a patient, the method comprising:
providing a medical device comprising:
a cannula defining a central channel extending from a proximal end to a distal end of the cannula, the cannula defining a lateral port proximate the distal end and in communication with the central channel, the cannula further comprising a sharp distal tip;
a handle disposed at the cannula proximal end, the handle comprising a first actuator;
a stylet disposed within the central channel of the cannula, the stylet defining a proximal end and a distal end, wherein the stylet is coupled with the first actuator and axially movable, in response to actuation of the first actuator, such that the distal end of the stylet is advanceable out of the lateral port and retractable into the central channel of the cannula; and
a cannulated probe disposed within the central channel of the shaft and surrounding at least a portion of the stylet, the cannulated probe defining a proximal end and a distal end, the cannulated probe further comprising a pair of electrodes, wherein the cannulated probe axially movable, in response to actuation of the second actuator, such that the distal end of the cannulated probe is advanceable out of the lateral port and retractable into the central channel of the cannula;
inserting the cannula of the medical device through cortical bone of the vertebral body into a cancellous bone region of the vertebral body;
actuating the first actuator to advance the distal end of the stylet out of the lateral port and through the cancellous bone region to a treatment location proximate the basivertebral nerve;
via the pair of electrodes, applying radiofrequency energy to the treatment location to treat the basivertebral nerve.

18. The method of claim 17, wherein at least the distal end of the stylet has a preformed curvature such that, as the stylet is advanced out of the lateral port defined in the shaft of the medical device, the stylet curves away from a central axis of the shaft of the medical device.

19. The method of claim 17, wherein the operation of actuating the first actuator comprises rotating a thumbwheel.

20. The method of claim 17, wherein:
the handle further comprises a second actuator and the cannulated probe is coupled with the second actuator; and
the method further comprises actuating the second actuator to advance the distal end of the cannulated probe over the channeling member out of the lateral port to the treatment location.
